(19) United States
(12) Patent Application Publication
(10) Pub. No.: US 2011/0230756 A1
(43) Pub. Date: Sep. 22, 2011

(54) FLUID FLOW ASSESSMENT

(75) Inventors: Carl Henrik Axel Odeen, Rannegatan (SE); Bruce Shawn Spottiswoode, Western Cape Province (ZA)

(73) Assignees: University of Cape Town, Cape Town (ZA); South African Medical Research Council, Cape Town (ZA)

(21) Appl. No.: 13/121,811

(22) PCT Filed: Sep. 30, 2009
(86) PCT No.: PCT/IB09/07007

§ 371 Date: Jun. 2, 2011

(30) Foreign Application Priority Data
Sep. 30, 2008 (ZA) 2008/08345

(51) Int. Cl. A61B 5/055

(52) U.S. Cl. 600/419

(57) ABSTRACT

A method of assessing fluid flow in a body is provided which includes phase contrast velocity encoded MRI scanning the body to obtain the velocity of fluids flowing in each of a plurality of volume elements (voxels) in three orthogonal directions; determining whether the flow in each voxel (1) is significant typically by checking if it has a value exceeding a threshold value selected from either or both of a noise level value and a minimum expected constant flow or flow-time profile; comparing each voxel with significant flow to each adjacent voxels (4, 6, 6a, 8, 8a) in the same and adjacent parallel plains; registering a connection where an adjacent voxel has significant flow; and clustering and depicting connected voxels visually by computing isosurfaces.

### FIELD OF THE INVENTION

This invention relates to method of assessing fluid flow connectivity in a body. The invention relates more particularly, but not exclusively, to a method of assessing the flow of cerebrospinal fluid (CSF) in the human body using magnetic resonance imaging (MRI) and a suitable processor such as a computer.

The term “fluid” shall have its widest meaning in this specification and does not relate solely to CSF. Also, while the method of the invention is particularly aimed at fluid flow assessment in the human body, it can be applied to any suitable body, including animal bodies, industrial and medical devices.

### BACKGROUND TO THE INVENTION

Many techniques exist for imaging or measuring fluid flow in a body. These techniques may be either direct or indirect.

The CSF system in the human brain is complex and CSF flow has both pulsatile and non-pulsatile components. Obstructions in one or more of the CSF flow channels can have devastating effects, and current methods to assess these obstructions are invasive or offer limited information, or both. These include radionuclide cisternography and air-encephalography, both of which pose a risk of infection associated with the lumbar puncture. Furthermore, raised intracranial pressure may cause cerebral herniation if a lumbar puncture is performed on a patient with non-communicating hydrocephalus. Computed tomography (CT) is routinely used to visualise anatomy but the clinical interpretation is qualitative.

Blood flow can be qualitatively measured by injecting contrast agents and imaging with MRI, digital subtraction angiography, or CT. Non invasive time-of-flight MRI techniques also exist for imaging blood flow, but these are again qualitative and limited to unidirectional flow systems, Doppler ultrasound provides a non-invasive and quantitative measurement of fluid velocity, but imaging windows are limited and flow measurements are constrained to the direction parallel to the travelling ultrasound waves. Blood flow is typically pulsatile in the arterial system and non-pulsatile in the venous system. The pulsatility is not central to this invention.

Phase contrast (PC) MRI quantitatively measures flow by encoding the velocity of the flowing fluid into the phase of the MRI signal. In clinical practice, 2D slices are typically imaged with flow encoded in through-plane or in-plane directions. This has limitations in that only selected 2D windows are used to examine an often complex 3D flow system. If the 2D slices are not very carefully selected the resultant image will not necessarily be useful in showing blockages and/or anastomoses.

Recently, MRI PC time-resolved flow sequences have evolved where a 3D volume is imaged with velocity encoded in three orthogonal directions. These techniques have predominantly been used to measure regional blood flow. In a technique known as phase contrast angiography, the magnitude and phase data have also been combined to yield 3D volume angiograms thus portraying detailed vessel structure without the need for MRI contrast agents [See references 1,2 below]. However, the inclusion of magnitude information in these angiograms detaches the result from the underlying flow, which is contained in the phase information. Technological advances have resulted in a rapid reduction in MRI acquisition time. Furthermore, wide-bore scanners and moving table MRI allow for an ever-increasing field of view. Careful visual analysis of complex flow systems will become increasingly tedious and time consuming as this technology evolves.

### OBJECT OF THE INVENTION

It is an object of this invention to provide a method of rapidly and automatically assessing 3D fluid flow connectivity which will at least partially alleviate some of the abovementioned problems. It is another object of the invention to provide a technique aimed at identifying a complex 3D volume of flowing fluid from an expected flow signature, and using 3D clustering/connectivity algorithms to automatically identify flow blockages or anastamoses.

### SUMMARY OF THE INVENTION

In accordance with this invention there is provided a method of assessing fluid flow in a body which includes
phase contrast velocity encoded MRI scanning the body to obtain the velocity of fluids flowing in each of a plurality of volume elements (voxels) in three orthogonal directions,
determining whether the flow in each voxel is significant,
comparing each voxel with significant flow to each of a number of adjacent voxels and registering a connection where an adjacent voxel has significant flow,
clustering and depicting connected voxels.

Further features of the invention provide for the clustered connected voxels to be visually depicted by computing isosurfaces from the clusters; and for the largest isosurface of connected voxels to be depicted.

Yet further features of the invention provide for the flow in a voxel to be significant if it has a value exceeding a threshold value selected from a noise level value and a minimum expected constant flow, a noise level value and a minimum expected pulsatile flow, a pre-determined flow-time profile, and a pre-determined periodicity constraint; and for the noise level to be determined by analysing histograms of stationery tissue and flow containing regions.

In phase contrast velocity encoding, the magnitude of the complex MRI signal is proportional to the MR signal of the material/fluid being imaged, and the phase is proportional to the velocity of the material/fluid. Recent MRI techniques allow a 3D volume to be scanned with three orthogonal velocity measurements at each voxel, and time-resolved through the cardiac cycle.

Still further features of the invention provide for the scan phase data to be pre-processed, such pre-processing to include phase unwrapping and background phase correction; and for an integrated flow volume to be

obtained for each voxel according to the formula:

$$V = {\sum }_{n = 1}^{N} \sqrt{{X}_{n}^{2} + {Y}_{n}^{2} + {Z}_{n}^{2}} ,$$

where N is the total number of time points and Xₙ, Yₙ, and Zₙ, correspond to the 3D volumes for the three encoding directions at time point n.

Yet further features of the invention provide for each voxel with significant flow to be compared with at least four, preferably eight, adjacent voxels in the same plane and at least one, preferably nine, adjacent voxels in each of two parallel adjacent planes.

### BRIEF DESCRIPTION OF THE DRAWINGS

The invention will be described, by way of example only, with reference to the drawings in which:

FIG. 1 is an exploded schematic representation of voxel comparison; and

FIG. 2 is a 3 dimensional illustration of an isosurface obtained from voxel flow information (the three dimensional effect being diminished by the use of the colors white and black).

### DETAILED DESCRIPTION WITH REFERENCE TO THE DRAWINGS

According to one embodiment of the invention, CSF flow is assessed in the body by initially using PC MRI to encode the velocity of flowing fluids into the phase of the MRI signal. A three dimensional (3D) PC MRI scan of the patient's head is performed with velocity encoded in three orthogonal directions, X, Y and Z for each voxel making up the patient's head. In this embodiment, each voxel is 1.5 mm³. The scan is prospectively or retrospectively gated to the patient's simultaneously measured electrocardiogram (ECG), and multiple time points are acquired covering the majority of the cardiac cycle. This gating allows one to measure dynamic periodic flow patterns.

The data is then pre-processed. This includes spatio-temporal phase unwrapping and correction of phase inhomogeneities. Hereafter the velocity data from the three encoding directions is combined to create an integrated flow volume for each voxel according to the formula

$$V = {\sum }_{n = 1}^{N} \sqrt{{X}_{n}^{2} + {Y}_{n}^{2} + {Z}_{n}^{2}} ,$$

where N is the total number of time points and Xₙ, Yₙ, and Zₙ correspond to the 3D volumes for the three velocity encoding directions at time point n. This serves to highlight voxels containing flow. It is to be noted that only the phase data is used; unlike 3D PC MRI angiograms, the magnitude data is ignored completely.

A threshold is then selected from either one or a combination of a noise level value, a minimum flow value. The noise level is determined by analysing histograms of stationery tissue and flow containing regions, whilst minimum flow is calculated based on the expected flow profile for the fluid. Voxels with flow above the threshold are indicated as having significant flow as a binary value. This results in a 3D binary image representing regions with significant flow.

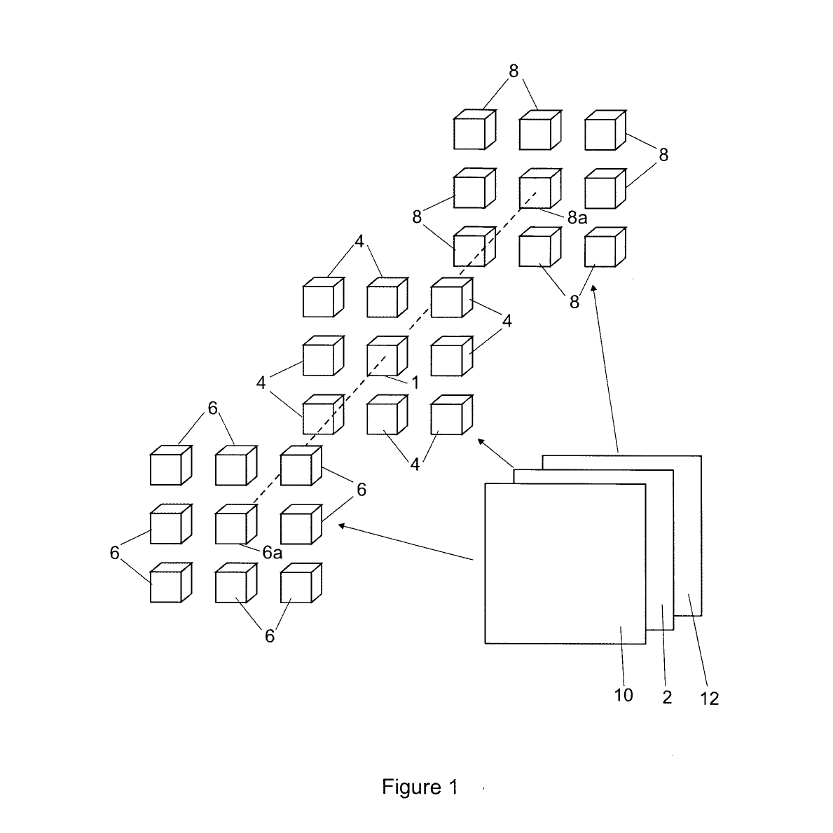

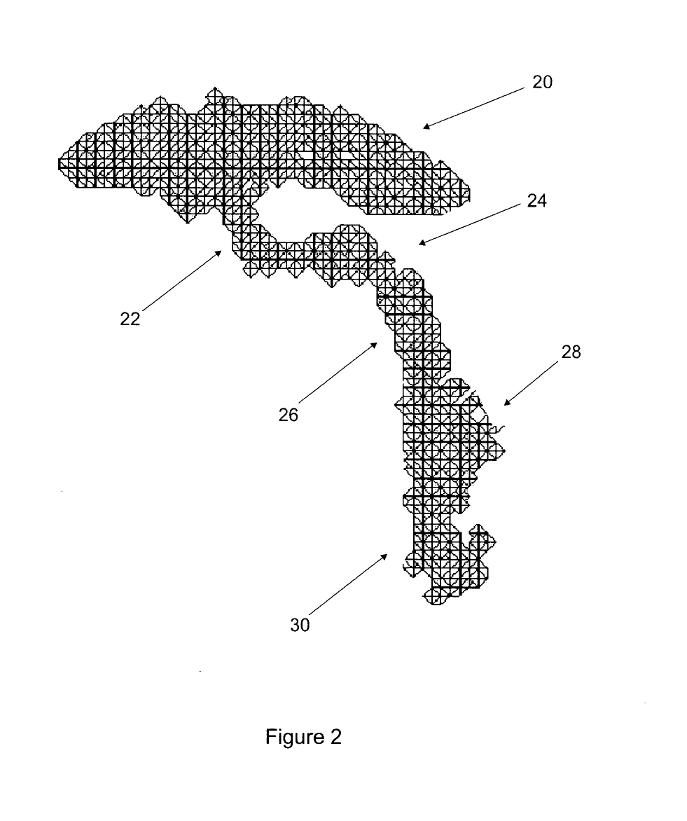

A 3D connectivity analysis is subsequently performed on each voxel having significant flow. In terms of this process each voxel with significant flow is compared to each of a plurality of adjacent voxels (often referred to as “nearest neighbour analysis”). FIG. 1 is an exploded diagram of voxels in three adjacent parallel planes and assists in illustrating this process. As shown, a voxel (1) in a first plane (2) is surrounded by eight other adjacent voxels (4). It is also adjacent nine voxels (6, 8) in each adjacent parallel plane (10, 12). This totals twenty six adjacent voxels and it is preferred that the voxel (1) be compared to all twenty six. However, the voxel (1) should at least be compared to four voxels in the same plane (2), one on each side, and at least the directly adjacent voxel (6a, 8a) in the adjacent planes (10, 12).

Where the adjacent voxel has significant flow a connection is registered. The connected voxels are then clustered and visually depicted. This is conveniently done by computing isosurfaces from the clusters. Typically only the largest connected region of voxels is depicted as an isosurface, but any suitable isosurface could be used. Such a region for a CSF system is shown in FIG. 2, where flow connectivity is demonstrated from the lateral ventricles part of which are indicated by numeral (20); through the foramen of Monroe indicated by numeral (22); through the Third Ventricle indicated by numeral (24); through the Aqueduct of Sylvius indicated by numeral (26); through the Fourth Ventricle indicated by numeral (28); to below the foramen of Magendie indicated by numeral (30). It will be appreciated that the 3D image can be viewed from any suitable perspective on the monitor of the processing system. This is useful in discerning between communicating and non-communicating hydrocephalus.

The method of the invention thus permits a purely flow-based 3D isosurface image illustrating a volume in which significant flow occurs. It also allows, for example, the whole CSF system to be examined in a single scan which simplifies the assessment of an occlusion's position and severity.

Since only the largest connected region of voxels is shown it is easy to determine if there are any occlusions or blockages along the pathways. This technique is useful when examining and accessing various diseases and medical conditions, for example hydrocephalus and Chiari malformation. It can also be used post-surgery to validate whether, for example, a third ventriculostomy has achieved the desired result. The technique could forseeably also be used to check flow velocities in shunts, used for pressure relief in hydrocephalus patients.

It will be appreciated that the technique of the invention could also be applied to any 3D PC MRI flow imaging application. In particular, it could be applied to vascular imaging with 3D PC MRI and to non-pulsatile flows.

Also, many other embodiments of the method exist which fall within the scope of the invention, particularly regarding the 3D PC MRI sequence, and manner in which significant flow is determined. For example, the threshold could also include a particular flow signature [see reference 3], and for dynamic flow, measures of periodicity of specific flow signatures [see reference 4] may also be used to dichotomise significant and non-significant flow.

The techniques described in references [3] and [4] were developed for 2D scans and make no mention of extension to 3D. The extension of [3] to 3D requires further adaptation as both CSF and vascular flow systems have different flow profiles depending on the position within the flow system. In reference [3] a flow signature is cross-correlated with each pixel in a 2D image. If the technique were extended to 3D then the aforementioned flow signature would need to correlated repeatedly in the 3D volume after being repeatedly scaled and phase-shifted within physiological limits.

Of course, the voxel size may be very as required and according to the processing power of the equipment used, as will be quite apparent to those skilled in the art.

### REFERENCES

1. Bock J, Wieben O, Johnson K M, Hennig J, Markl M. Optimal processing to derive static PC-MRA from time-resolved 3D PC-MRI data. Proc. Intl. Soc. Mag. Reson. Med. 2008;16:3053.
2. Anderson A G, Johnson K M, Bock J, Markl M, Wieben O. Comparison of Image Reconstruction Algorithms for the Depiction of Vessel Anatomy in PC VIPR Datasets. Proc. Intl. Soc. Mag. Reson. Med. 2008;16:934.
3. Alperin N and S H Lee. PUBS: Pulsatility-Based Segmentation of Lumens Conducting Non-steady Flow. Magnetic Resonance in Medicine 2003; 49:934-944.
4. Balédent O, Henry-Feugeas M-C C, Idy-Peretti I. Cerebrospinal fluid dynamics and relation with blood flow. A magnetic resonance study with semiautomated cerebrospinal fluid segmentation. Investigative Radiology 2001;36(7):368-377.

1. A method of assessing fluid flow in a body which includes phase contrast velocity encoded MRI scanning the body to obtain the velocity of fluids flowing in each of a plurality of volume elements (voxels) in three orthogonal directions, determining whether the flow in each voxel is significant, comparing each voxel with significant flow to each of a number of adjacent voxels and registering a connection where an adjacent voxel has significant flow, and clustering and depicting connected voxels.

2. A method of assessing fluid flow in a body as claimed in claim 1 wherein the clustered connected voxels are visually depicted by computing isosurfaces from the clusters.

3. A method of assessing fluid flow in a body as claimed in claim 2 wherein the largest isosurface of connected voxels is depicted.

4. A method of assessing fluid flow in a body as claimed in claim 1 wherein the flow in a voxel is significant if it has a value exceeding a threshold value selected from a noise level value and a minimum expected constant flow, a noise level value and a minimum expected pulsatile flow, a pre-determined flow-time profile, and a pre-determined periodicity constraint.

5. A method of assessing fluid flow in a body as claimed in claim 4 wherein the noise level is determined by analysing histograms of stationery tissue and flow containing regions.

6. A method of assessing fluid flow in a body as claimed in claim 1 wherein the scan phase data is pre-processed, such pre-processing including phase unwrapping and background phase correction.

7. A method of assessing fluid flow in a body as claimed in claim 1 wherein an integrated flow volume is obtained for each voxel according to the formula:
$$V = {\sum }_{n = 1}^{N} \sqrt{{X}_{n}^{2} + {Y}_{n}^{2} + {Z}_{n}^{2}} ,$$
where N is the total number of time points and Xₙ, Yₙ, and Zₙ correspond to the 3D volumes for the three encoding directions at time point n.

8. A method of assessing fluid flow in a body as claimed in claim 1 wherein each voxel with significant flow is compared with at least four adjacent voxels in the same plane and at least one adjacent voxel in each of two parallel adjacent planes.

9. A method of assessing fluid flow in a body as claimed in claim 8 wherein each voxel with significant flow is compared with eight adjacent voxels in the same plane and nine adjacent voxel in each of two parallel adjacent planes.

10. A method of assessing fluid flow in a body as claimed in claim 2 wherein the flow in a voxel is significant if it has a value exceeding a threshold value selected from a noise level value and a minimum expected constant flow, a noise level value and a minimum expected pulsatile flow, a pre-determined flow-time profile, and a pre-determined periodicity constraint.

11. A method of assessing fluid flow in a body as claimed in claim 3 wherein the flow in a voxel is significant if it has a value exceeding a threshold value selected from a noise level value and a minimum expected constant flow, a noise level value and a minimum expected pulsatile flow, a pre-determined flow-time profile, and a pre-determined periodicity constraint.

12. A method of assessing fluid flow in a body as claimed in claim 10 wherein the noise level is determined by analysing histograms of stationery tissue and flow containing regions.

13. A method of assessing fluid flow in a body as claimed in claim 11 wherein the noise level is determined by analysing histograms of stationery tissue and flow containing regions.

14. A method of assessing fluid flow in a body as claimed in claim 2 wherein the scan phase data is pre-processed, such pre-processing including phase unwrapping and background phase correction.

15. A method of assessing fluid flow in a body as claimed claim 3 wherein the scan phase data is pre-processed, such pre-processing including phase unwrapping and background phase correction.

16. A method of assessing fluid flow in a body as claimed in claim 4 wherein the scan phase data is pre-processed, such pre-processing including phase unwrapping and background phase correction.

17. A method of assessing fluid flow in a body as claimed in claim 5 wherein the scan phase data is pre-processed, such pre-processing including phase unwrapping and background phase correction.

18. A method of assessing fluid flow in a body as claimed in claim 10 wherein the scan phase data is pre-processed, such pre-processing including phase unwrapping and background phase correction.

19. A method of assessing fluid flow in a body as claimed in claim 11 wherein the scan phase data is pre-processed, such pre-processing including phase unwrapping and background phase correction.

20. A method of assessing fluid flow in a body as claimed in claim 12 wherein the scan phase data is pre-processed, such pre-processing including phase unwrapping and background phase correction.

21. A method of assessing fluid flow in a body as claimed in claim 13 wherein the scan phase data is pre-processed, such pre-processing including phase unwrapping and background phase correction.
